(19) United States
(12) Patent Application Publication
(10) Pub. No.: US 2024/0167943 A1
(43) Pub. Date: May 23, 2024

(54) METHOD AND SYSTEM FOR DETERMINING SUPPORT STRUCTURE BY COMBINING STRESS ENVIRONMENT WITH UNDERGROUND SURROUNDING ROCK STRUCTURE

(71) Applicants: Guizhou University, Guiyang (CN); SHANDONG UNIVERSITY OF SCIENCE AND TECHNOLOGY, Qingdao (CN)

(72) Inventors: Zhijie WEN, Guiyang (CN); Zhenqi Song, Qingdao (CN); Yujing Jiang, Qingdao (CN); Shucai Li, Jinan (CN); Yujun Zuo, Guiyang (CN); Jianping Zuo, Beijing (CN); Kang Peng, Changsha (CN); Shankun Zhao, Beijing (CN)

(21) Appl. No.: 18/505,930

(22) Filed: Nov. 9, 2023

(30) Foreign Application Priority Data
Nov. 22, 2022 (CN) 202211467266.8

(51) Int. Cl. G01N 21/31

(52) U.S. Cl. CPC G01N 21/31; G01N 2201/06113; G01N 2201/0636

(57) ABSTRACT

A method and a system for determining a supporting structure by combining a stress environment and an underground surrounding rock structure are provided in the disclosure, which relates to the technical field of stability analysis of coal-mine rock mass. The method includes: defining a stress peak position and an in-situ stress position by determining the stress environment; identifying the underground surrounding rock structure, identifying lithology and constructing a three-dimensional model of a rock stratum to analyze damage degree of the rock stratum; pretreating, namely normalizing, the damage degree of the rock stratum at two sides and comparing the damage degree of the rock stratum at a roof and the floor; and identifying the supporting structure and determining supporting effectiveness and a supporting length. The method of the disclosure is different from related art, and ensures that the supporting structure meets mechanical foundation and practical engineering requirements as a whole.

### CROSS-REFERENCE TO RELATED APPLICATIONS

This application claims priority to Chinese Application No. 202211467266.8, filed on Nov. 22, 2022, entitled “Method and system for determining supporting structure by combining stress environment with underground surrounding rock structure”. These contents are hereby incorporated by reference.

### TECHNICAL FIELD

The disclosure relates to the technical field of stability analysis of coal-mine rock mass, in particular to a method and a system for determining a supporting structure by combining a stress environment and an underground surrounding rock structure.

### BACKGROUND ART

Stability analysis of rock mass has always been an important issue in mining engineering. Currently, a method for the stability analysis of rock mass mainly includes a rigid body limit equilibrium method, a numerical method or analytical method, a reliability analysis method, etc. The first two methods are mainstream analysis methods at present, in which results can be obtained through statistical analysis based on experience of engineering practice, with characteristics of being fast and easy to master. However, due to imprecision in an empirical production process, needs of practical engineering cannot be fully met. Meanwhile, the mainstream analysis methods at present lack overall analysis of a stress environment, a surrounding rock structure and a supporting structure and seize on accurate and complicated mathematical calculation, which results in large difference between theoretical assumptions for numerical calculation and actual rock mass, and is difficult to involve construction conditions that have great impact on stability of the rock mass in the calculation. Therefore, following aspects should be considered in a method for stability analysis of the rock mass: complexity of underground engineering, integrity of factors affecting stability of the rock mass and limitations of basic theoretical researches.

At present, analysis of stability of the rock mass in existing technologies cannot meet needs of overall, practical, convenient and comprehensive analysis of the stability of the rock mass, so it is necessary to propose a new, systematic and practical method and device for stability analysis of the rock mass.

### SUMMARY

One of objects of the disclosure is to provide a method for determining a supporting structure by combining a stress environment with an underground surrounding rock structure, which jointly determines the supporting structure using analysis and processing results of the stress environment and the underground surrounding rock structure, so as to ensure that the supporting structure meets mechanical foundation and actual engineering requirements.

In order to achieve the above object, the disclosure adopts following technical schemes.

The disclosure relates to a method for determining a supporting structure by combining a stress environment and an underground surrounding rock structure, which includes following steps:

a, determining the stress environment where a rock stratum around an underground space is located and defining a peak stress position and an in-situ stress position;

b, intelligently identifying the underground surrounding rock structure by using a laser instrument, identifying lithology and a thickness of the rock stratum around the underground space, constructing a three-dimensional model of the rock stratum so as to analyze damage degree of the rock stratum at two sides, a roof and a floor of the underground surrounding rock structure, at the peak stress position and the in-situ stress position;

c, normalizing the damage degree of the rock stratum at the two sides, and comparing the damage degree of the rock stratum at the roof and at the floor; and

d, determining supporting effectiveness by combining the stress environment and the underground surrounding rock structure.

The above technical scheme directly brings beneficial technical effects as follows.

In the technical scheme, firstly, the stress peak position and the in-situ stress position are defined by determining the stress environment. Then, the underground surrounding rock structure is identified, the lithology is identified using the laser instrument, and the three-dimensional model of the rock stratum is constructed to analyze the damage degree of the rock stratum. Following this, the damage degree of the rock stratum at the two sides is pretreated, namely normalized, and the damage degree of the rock stratum at the roof and the floor is compared. Finally, a reinforcement manner is determined, and the supporting effectiveness and supporting length are determined according to the stress environment and the underground surrounding rock structure.

The above technical scheme, as a whole, mainly provides a new method to determine the supporting structure by using the stress environment and the underground surrounding rock structure, which is different from a rigid body limit equilibrium method, a numerical method or analytical method, a reliability analysis method in related art. With the above technical scheme, it is ensured that the supporting structure meets mechanical foundation and practical engineering requirements as a whole.

For the method for determining the supporting structure by combining the stress environment with the underground surrounding rock structure, and in step a, the stress environment where a rock stratum around an underground space is located is determined by using numerical analysis or elastoplastic mechanic calculation; and in step c, the normalizing includes following steps.

Firstly, a fracture state and a number of fractures are analyzed according to the damage degree of the rock stratum at different positions, and the damage degree of the rock stratum is numerically expressed by a function.

Then, a maximum value and a minimum value of the damage degree b of the rock stratum are obtained. The damage degree of the rock stratum at the in-situ stress position is the maximum value, and the damage degree of the rock stratum at the two sides is the minimum value. Then, original data of the damage degree of the rock stratum at respective positions are linearly transformed to cause all data to fall within an interval of [0, 1], with a transformation function shown in formula (1):

$$a = \frac{b - {b}_{min}}{{b}_{max} - {b}_{min}} \qquad ( 1 )$$

In the formula (1), a represents a numerical value of the damage degree of the rock stratum after normalization; and b represents the original value of the damage degree of the rock stratum.

Finally, normalized data of the damage degree of the rock stratum is obtained, and the data is an infinite value in a distribution interval of [0, 1]. The larger the value, the smaller the damage degree of the rock stratum, and the smaller the value, the greater the damage degree of the rock stratum.

In step d, the determining the supporting effectiveness includes: determining whether existing supporting is effective and meets supporting requirements; obtaining deformation of the rock stratum in the underground space by software analysis, and comparing the deformation with a multiple of a size of a tunnel, a value of the multiple of the size of the tunnel being obtained according to use of the underground space and analysis of rock lithology; determining the existing supporting to be effective if the deformation is less than the multiple of the size of the tunnel; and determining the existing supporting to be ineffective and requiring changing of a supporting manner if the deformation is equal to or greater than the multiple of the size of the tunnel; and determining a supporting length according to the stress environment and the underground surrounding rock structure after the supporting effectiveness is determined.

Further, the laser instrument includes a shell, and a laser spectroscopy device, a rotating device and a laser scanning device integrally arranged in the shell. The rotating device is configured for rotating the laser scanning device, the laser scanning device is located above the rotating device, and the laser spectroscopy device is located below the rotating device. In actual use, the laser instrument is equipped with a telescopic rod and a placing frame. A front end of the telescopic rod is connected to the laser instrument, and a rear end of the telescopic rod is connected to the placing frame, so that the laser instrument can enter an inner wall of a bore hole for detection by adjusting the telescopic rod to obtain the lithology and the thickness of the rock stratum. A rotating speed of the rotating device is matched with a displacement speed of the telescopic rod.

Further, the laser spectroscopy device includes a laser source, a focusing lens, a reflecting mirror and a grating. The laser source is configured for reflecting high-energy pulsed laser. The focusing lens is configured for improving capability for detection of the laser at an edge and increasing energy of the laser. The reflecting mirror is configured to refract the high-energy pulsed laser so as to irradiate on a rock surface.

Further, in step d, a length of an anchor rod for supporting is selected at an interval [0, ½] for the normalized damage degree.

Further, in the step d, when the supporting is ineffective and the supporting manner needs to be changed, supporting density is determined according to a principle that energy E required for prestressing is equal to energy required for multiples of deformation of the underground space, and a value of the multiples of deformation of the underground space is obtained according to the use of the underground space and the analysis of rock lithology.

Further, in step c, comparing the damage degree of the rock stratum at the roof and at the floor is made by selecting rock stratum at different positions at the roof and the floor for comparison according to different lithology.

Further, the laser scanning device is located above the laser spectroscopy device. The laser scanning device includes a laser transmitter, a rotatable cylindrical filter, a receiver, a time counter and a CCD camera. The rotating device is located at an end of the laser scanning device close to the laser spectroscopy device, the laser transmitter is configured for emitting infrared laser, and the rotatable cylindrical filter is configured for increasing a laser scanning area, and the laser is reflected back after scanning to the rock. The receiver is configured for receiving a scanning rate. The CCD camera is configured for shooting the rock stratum, and the time counter is configured for controlling the scanning rate. The laser scanning device can rotate 360 degrees under action of the rotating device.

Another object of the present disclosure is to provide a system for identifying an underground surrounding rock structure by using the method for determining the supporting structure by combining the stress environment and the underground surrounding rock structure, which includes a computer, a data transmission line and a laser instrument. The laser instrument includes a shell, and a laser spectroscopy device, a rotating device and a laser scanning device integrally arranged in the shell. The rotating device is configured for rotating the laser scanning device, the laser scanning device is located above the rotating device, and the laser spectroscopy device is located below the rotating device. The laser instrument can extend into an inner wall of a bore hole for detection to obtain the lithology and the thickness of the rock layer.

The laser scanning device is located above the laser spectroscopy device. The laser scanning device includes a laser transmitter, a rotatable cylindrical filter, a receiver, a time counter and a CCD camera. The rotating device is located at an end of the laser scanning device close to the laser spectroscopy device, the laser transmitter is configured for emitting infrared laser, and the rotatable cylindrical filter is configured for increasing a laser scanning area, and the laser is reflected back after scanning to the rock. The receiver is configured for receiving a scanning rate. The CCD camera is configured for shooting the rock stratum, and the time counter is configured for controlling the scanning rate. The laser scanning device can rotate 360 degrees under action of the rotating device.

The laser spectroscopy device includes a laser source, a focusing lens, a reflecting mirror and a grating. The laser source is configured for reflecting high-energy pulsed laser. The focusing lens is configured for improving capability for detection of the laser at an edge and increasing energy of the laser. The reflecting mirror is configured to refract the high-energy pulsed laser so as to irradiate on a rock surface. Excited rock photons are collected by the laser instrument and are transmitted to the computer through the data transmission line, and a spectral image of the rock is formed by software processing in the computer, which is analyzed and compared with data in the database, and a lithology name and spectral characteristics are displayed on a computer screen.

Compared with the related art, the disclosure has following beneficial technical effects.

(1) The method for determining the supporting structure is provided in this disclosure, which is different from the relate art. In this method, the laser instrument is adopted to intelligently identify the underground surrounding rock structure. Lithology identification and three-dimensional modeling can be made simultaneously through cooperation of the laser spectroscopy device and the laser scanning device in the laser instrument. The rotating speed of the rotating device in the laser instrument is regulated according to a proceeding speed of the telescopic rod, so that full-coverage scanning of a surface of the inner wall of the bore hole can be ensured, and detection accuracy of the laser instrument can be improved.

(2) The laser spectroscopy device in the laser instrument of the disclosure can quickly transmit data to a microcomputer after the excited rock photons are collected, and a spectrum image of the rock can be formed by processing from software, and then analyzed and compared with the data in the database, and finally the lithology name and spectrum characteristics can be displayed, and the focusing lens inside the laser spectroscopy device can meet requirements of practical application. The CCD camera inside the laser scanning device can generate color images of various environments.

(3) With mutual cooperation of the laser instrument, the placing frame and the telescopic rod, the laser instrument can enter the inner wall of the bore hole for detection by adjusting the telescopic rod to obtain the lithology and thickness of the rock stratum.

(4) In order to eliminate inaccuracy for various evaluation criteria (such as dimensional influence among uniaxial strength, triaxial strength and deformation modulus) in analyzing the damage degree of the rock stratum, in the disclosure, the original data can compared after the data is normalized, and the data is dimensionless data after normalization. In addition, another purpose of normalizing is to ensure that external influence is eliminated within a certain range after the original data is processed, so as to improve accuracy in evaluating the damage.

(5) In the method for determining the supporting structure by combining the stress environment and the surrounding rock structure according to the disclosure, the supporting structure can be jointly determined according to the stress environment and the underground surrounding rock structure, which not only follows principles of safety, economy, rationality and suitability for site of the supporting structure, but also performs overall analysis of three factors affecting stability of the rock mass.

### BRIEF DESCRIPTION OF THE DRAWINGS

The present disclosure will be further described with reference to the accompanying drawings.

FIG. 1 is a flow chart of a method for determining a supporting structure according to the present disclosure;

FIG. 2 is a sectional view of a laser instrument according to the present disclosure; and

FIG. 3 is a schematic diagram of a use state of a laser instrument according to the present disclosure;

### REFERENCE NUMBERS ARE AS FOLLOWS

1. Rotatable Cylindrical Filter, 2. Receiver, 3. Laser Transmitter, 4. CCD Camera, 5. Time Counter, 6. Rotating Device, 7. Reflecting Mirror, 8. Focusing Lens, 9. Grating, 10. Laser Source, 11. Telescopic Rod, 12. Placing Frame, 13. Bore Hole, 14. Laser Instrument.

### DETAILED DESCRIPTION

The disclosure will be further described below with reference to the accompanying drawings and embodiments.

A method and a system for determining a supporting structure by combining a stress environment and an underground surrounding rock structure are provided in this disclosure.

A main technical idea of the disclosure lies in that the supporting structure is determined by using the stress environment and the underground surrounding rock structure, and the supporting structure can be determined on a basis of basic theoretical mechanics by analyzing a relationship between measured values and construction conditions and using analysis and processing results of the stress environment and the underground surrounding rock structure fused with actual underground engineering status. During identifying of the surrounding rock structure, a new laser instrument 14 is adopted, which can simultaneously complete lithology identification and three-dimensional modeling. Compared with the related art, with the method for determining the supporting structure according to this disclosure, it can be ensured that the supporting structure meets requirements of mechanical foundation and practical engineering, and gets rid of constraints of empirical and numerical analysis.

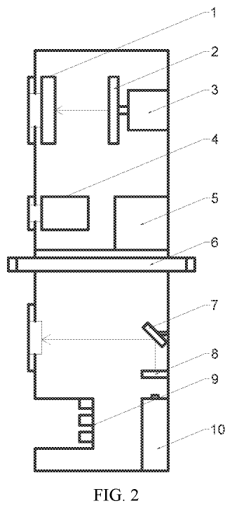

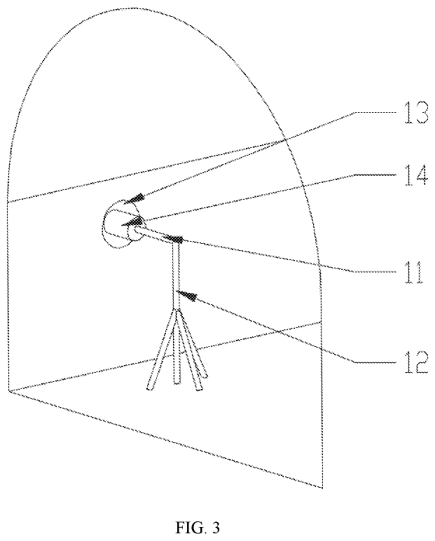

As shown in FIGS. 2 to 3, the laser instrument 14 of the present disclosure includes a shell, and a laser spectroscopy device, a rotating device 6 and a laser scanning device integrally arranged in the shell. The rotating device is configured for rotating the laser scanning device, the laser scanning device is located above the rotating device, and the laser spectroscopy device is located below the rotating device.

The laser scanning device includes a laser transmitter 3, a rotatable cylindrical filter 1, a receiver 2, a time counter 5 and a CCD camera 4. The rotating device 6 is located at an end of the laser scanning device close to the laser spectroscopy device, the laser transmitter 3 is configured for emitting infrared laser, and the rotatable cylindrical filter 1 is configured for increasing a laser scanning area, and the laser is reflected back after scanning to the rock. The receiver 2 is configured for receiving a scanning rate. The CCD camera 4 is configured for shooting the rock stratum, and the time counter is configured for controlling the scanning rate. The laser scanning device can rotate 360 degrees under action of the rotating device. When the laser scanning device is operated, the laser transmitter emits infrared laser, and the laser scanning area is increased through the rotatable cylindrical filter. After scanning to the rock, the laser is reflected back, and scanning data is received through the receiver. At the same time, the CCD camera shoots the rock in real time so as to obtain a real rock image, and the time counter controls the scanning rate so as to cooperate with a rotation of the rotating device to improve scanning accuracy. A rotating speed of the rotating device of the laser scanning device is matched with a displacement speed of the telescopic rod to ensure that the scanning device can scan an inner wall of the bore hole in all aspects. The placing frame is equipped with an automatic alignment system, which can align a central axis of the telescopic rod with a central axis of the bore hole to ensure accuracy of the instrument.

The laser spectroscopy device includes a laser source 10, a focusing lens 8, a reflecting mirror 7 and a grating 9. The laser source 10 is configured for reflecting high-energy pulsed laser. The focusing lens 8 is configured for improving capability for detection of the laser at an edge and increasing energy of the laser. The reflecting mirror is configured to refract the high-energy pulsed laser so as to irradiate on a rock surface. When the device is operated, the laser source emits high-energy pulsed laser, which passes through a focusing lens of an optical system to improve capability for detection of the laser at an edge and increasing energy of the laser. The reflecting mirror then refracts the high-energy pulsed laser so as to irradiate on a rock surface.

As shown in FIG. 3, in actual use, the laser instrument is equipped with a telescopic rod 11 and a placing frame 12. A front end of the telescopic rod is connected to the laser instrument 14, and a rear end of the telescopic rod is connected to the placing frame, so that the laser instrument can enter an inner wall of a bore hole 13 for detection by adjusting the telescopic rod to obtain the lithology and the thickness of the rock stratum. The rotating speed of the rotating device is matched with the displacement speed of the telescopic rod. The telescopic rod and the placing frame are fixed together. The placing frame mainly facilitates fixing of the laser instrument, so a frame body of the placing frame is triangular, which can ensure its stability. The telescopic rod can for example be selected with a sleeve structure so as to assist in adjusting a position of the laser instrument.

Application of the laser instrument 14 in the method for determining the supporting structure according to the present disclosure has following advantages: (1) lithology identification and three-dimensional modeling of the rock mass can be completed at the same time. (2) The rotating speed of the rotating device is regulated according to the proceeding speed of the telescopic rod, so as to ensure full-coverage scanning of a surface of the inner wall of the bore hole. (3) During scanning of the laser scanning device, the laser spectroscopy device performs random detection in different areas for ten times, and emits pulsed laser for three times each time. When the laser spectroscopy device is operated, the telescopic rod stops moving, while the rotating device of the laser scanning device stops operation. After the laser spectroscopy device finishes operation, other devices start operating again. (4) The laser spectroscopy device can immediately transmit data to a microcomputer after the excited rock photons are collected, and a spectrum image of the rock can be formed by processing from software, and then analyzed and compared with the data in the database, and finally lithology name and spectrum characteristics can be displayed. (5) The laser spectroscopy device is a new laser instrument, with an internal focusing lens of an optical system meeting needs of practical application and an internal grating capable of accurately capturing photons of all kinds of rocks. (6) The laser scanning device is a new laser instrument, with an internal CCD camera capable of generating color images of various environments.

In the following, the method for determining the supporting structure by combining the stress environment and the underground surrounding rock structure according to the present disclosure will be illustrated as follows.

The method for determining the supporting structure by combining the stress environment and the underground surrounding rock structure includes following steps 1 to 4.

In step 1, the stress environment where a rock stratum around an underground space is located is determined and a peak stress position and an in-situ stress position is defined. The stress environment where the rock stratum around the underground space is located is determined by using numerical analysis or elastoplastic mechanic calculation.

In step 2, the underground surrounding rock structure is intelligently identified by using a laser instrument, lithology and a thickness of the rock stratum around the underground space are identified, a three-dimensional model of the rock stratum is constructed so as to analyze damage degree of the rock stratum at two sides, a roof and a floor of the underground surrounding rock structure, at the peak stress position and the in-situ stress position.

In step 3, the damage degree of the rock stratum at the two sides is normalized, and the damage degree of the rock stratum at the roof and at the floor is compared. The normalizing includes following steps.

Firstly, a fracture state and a number of fractures are analyzed according to the damage degree of the rock stratum at different positions, and the damage degree of the rock stratum is numerically expressed by a function.

Then, a maximum value and a minimum value of the damage degree b of the rock stratum are obtained. The damage degree of the rock stratum at the in-situ stress position is the maximum value, and the damage degree of the rock stratum at the two sides is the minimum value. Then, original data of the damage degree of the rock stratum at respective positions are linearly transformed to cause all data to fall within an interval of [0, 1], with a transformation function shown in formula (1):

$$a = \frac{b - {b}_{min}}{{b}_{max} - {b}_{min}} \qquad ( 1 )$$

In the formula (1), a represents a numerical value of the damage degree of the rock stratum after normalization; and b represents the original value of the damage degree of the rock stratum.

Finally, normalized data of the damage degree of the rock stratum is obtained, and the data is an infinite value in a distribution interval of [0, 1]. The larger the value, the smaller the damage degree of the rock stratum, and the smaller the value, the greater the damage degree of the rock stratum.

A step in which the damage degree of the rock stratum at the roof and at the floor is compared is made by selecting rock stratum at different positions at the roof and the floor for comparison according to different lithology.

In step 4, supporting effectiveness is determined by combining the stress environment and the underground surrounding rock structure, and main steps for determining the supporting effectiveness are as follows:

determining whether existing supporting is effective and meets supporting requirements;

obtaining deformation of the rock stratum in the underground space by software analysis, and comparing the deformation with a multiple of a size of a tunnel, a value of the multiple of the size of the tunnel being obtained according to use of the underground space and analysis of rock lithology; determining the existing supporting to be effective if the deformation is less than the multiple of the size of the tunnel; and determining the existing supporting to be ineffective and requiring changing of a supporting manner if the deformation is equal to or greater than the multiple of the size of the tunnel; and

determining a supporting length according to the stress environment and the underground surrounding rock structure after the supporting effectiveness is determined.

A length of an anchor rod for supporting is selected at an interval [0, ½] for the normalized damage degree.

When the supporting is ineffective and the supporting manner needs to be changed, supporting density is determined according to a principle that energy E required for prestressing is equal to energy required for multiples of deformation of the underground space, and a value of the multiples of deformation of the underground space is obtained according to the use of the underground space and the analysis of rock lithology.

In the following, the method for determining the supporting structure by combining the stress environment and the underground surrounding rock structure according to the present disclosure further be illustrated in combination with embodiments.

### EMBODIMENT 1:

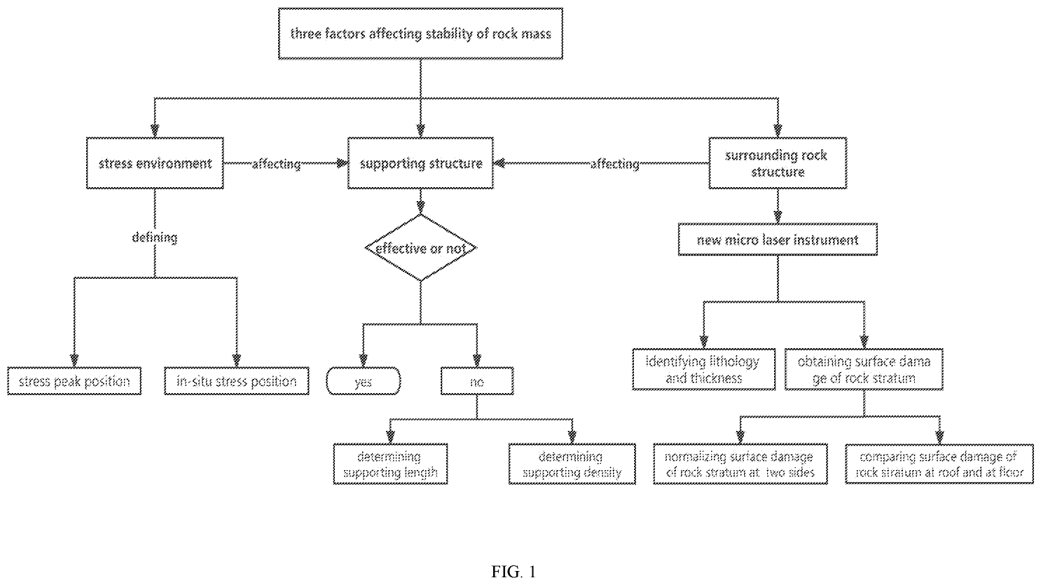

As shown in FIG. 1, the method for determining the supporting structure by combining the stress environment and the underground surrounding rock structure includes following steps 1 to 4.

In step 1, the stress environment of an underground space is determined by numerical analysis, elastoplastic mechanic calculation and other methods, and a peak stress position and an in-situ stress position is defined.

In step 2, lithology and a thickness are identified by using the laser instrument described above, and at the same time, a three-dimensional model of a rock stratum is constructed to analyze damage degree of the rock stratum at two sides, a roof and a floor of the underground surrounding rock structure, at the peak stress position.

In step 3, after data of the damage degree of the rock stratum is obtained, the damage degree of the rock stratum at the two sides is normalized, and the damage degree of the rock stratum at the roof and at the floor is compared. Firstly, a fracture state and a number of fractures are analyzed according to the damage degree of the rock stratum at different positions, and the damage degree of the rock stratum is numerically expressed by a function. Then, a maximum value and a minimum value of the damage degree of the rock stratum are obtained, the damage degree of the rock stratum at the in-situ stress position is the maximum value, and the damage degree of the rock stratum at the two sides is the minimum value. Then, original data of the damage degree of the rock stratum at respective positions are linearly transformed to cause all data to fall within an interval of [0, 1]. Finally, normalized data of the damage degree of the rock stratum is obtained, and the data is an infinite value in a distribution interval of [0, 1], the larger the value, the smaller the damage degree of the rock stratum, and the smaller the value, the greater the damage degree of the rock stratum. A step in which the damage degree of the rock stratum at the roof and at the floor is compared is made by selecting rock stratum at different positions at the roof and the floor for comparison according to different lithology.

In step 4, deformation of the rock stratum in the underground space is obtained by software analysis, and the deformation is compared with a multiple of a size of a tunnel, a value of the multiple of the size of the tunnel is obtained according to use of the underground space and analysis of rock lithology. Existing supporting is determined to be effective if the deformation is less than the multiple of the size of the tunnel; and the existing supporting is determined to be ineffective and changing of a supporting manner is required if the deformation is equal to or greater than the multiple of the size of the tunnel.

A supporting length is determined according to the stress environment and the underground surrounding rock structure after the supporting effectiveness is determined. A length of an anchor rod should be selected at an interval [0, ½] for the normalized damage degree. Supporting density is determined according to a principle that energy E required for prestressing is equal to energy required for multiples of deformation of the underground space, and a value of the multiples of deformation of the underground space is obtained according to use of the underground space and the analysis of rock lithology.

In the following, a system for identifying the underground surrounding rock structure by using the method for determining the supporting structure by combining the stress environment and the underground surrounding rock structure described above is described in detail. The system includes a computer, a data transmission line and a laser instrument. The laser instrument includes a shell, and a laser spectroscopy device, a rotating device and a laser scanning device integrally arranged in the shell. The rotating device is configured for rotating the laser scanning device, the laser scanning device is located above the rotating device, and the laser spectroscopy device is located below the rotating device. The laser instrument can extend into an inner wall of a bore hole for detection to obtain the lithology and the thickness of the rock layer.

The laser scanning device is located above the laser spectroscopy device. The laser scanning device includes a laser transmitter, a rotatable cylindrical filter, a receiver, a time counter and a CCD camera. The rotating device is located at an end of the laser scanning device close to the laser spectroscopy device, the laser transmitter is configured for emitting infrared laser, and the rotatable cylindrical filter is configured for increasing a laser scanning area, and the laser is reflected back after scanning to the rock. The receiver is configured for receiving a scanning rate. The CCD camera is configured for shooting the rock stratum, and the time counter is configured for controlling the scanning rate. The laser scanning device can rotate 360 degrees under action of the rotating device.

The laser spectroscopy device includes a laser source, a focusing lens, a reflecting mirror and a grating. The laser source is configured for reflecting high-energy pulsed laser. The focusing lens is configured for improving capability for detection of the laser at an edge and increasing energy of the laser. The reflecting mirror is configured to refract the high-energy pulsed laser so as to irradiate on a rock surface. Excited rock photons are collected by the laser instrument and are transmitted to the computer through the data transmission line, and a spectral image of the rock is formed by software processing in the computer, which is analyzed and compared with data in the database, and a lithology name and spectral characteristics are displayed on a computer screen.

Parts not described in the present disclosure can be realized by referring to the related art. The above are merely preferred embodiments of the present disclosure and are not intended to limit the present disclosure. The present disclosure may be subject to changes and variations for those skilled in the art. Any modifications, equivalent replacements, and improvements made within the spirit and principles of the present disclosure shall all be encompassed in the protection scope of the present disclosure.

1. A method for determining a supporting structure by combining a stress environment and an underground surrounding rock structure, comprising following steps:
a, determining the stress environment where a rock stratum around an underground space is located and defining a peak stress position and an in-situ stress position;
b, intelligently identifying the underground surrounding rock structure by using a laser instrument, identifying lithology and a thickness of the rock stratum around the underground space, constructing a three-dimensional model of the rock stratum so as to analyze damage degree of the rock stratum at two sides, a roof and a floor of the underground surrounding rock structure, at the peak stress position and the in-situ stress position;
c, normalizing the damage degree of the rock stratum at the two sides, and comparing the damage degree of the rock stratum at the roof and at the floor; and
d, determining supporting effectiveness by combining the stress environment and the underground surrounding rock structure; wherein
in step a, the stress environment where the rock stratum around the underground space is located is determined by using numerical analysis or elastoplastic mechanic calculation; and
in step c, the normalizing comprises:
analyzing a fracture state and a number of fractures according to the damage degree of the rock stratum at different positions, and the damage degree of the rock stratum being numerically expressed by a function;
obtaining a maximum value and a minimum value of the damage degree b of the rock stratum, the damage degree of the rock stratum at the in-situ stress position being the maximum value, and the damage degree of the rock stratum at the two sides being the minimum value; and linearly transforming original data of the damage degree of the rock stratum at respective positions to cause all data to fall within an interval of [0, 1], with a transformation function shown in formula (1):
$$a = \frac{b - {b}_{min}}{{b}_{max} - {b}_{min}} \qquad ( 1 )$$
where a represents a numerical value of the damage degree of the rock stratum after normalization; and b represents the original value of the damage degree of the rock stratum; and
obtaining normalized data of the damage degree of the rock stratum, the data being an infinite value in a distribution interval of [0, 1], the larger the value, the smaller the damage degree of the rock stratum, and the smaller the value, the greater the damage degree of the rock stratum;
in step d, the determining the supporting effectiveness comprises:
determining whether existing supporting is effective and meets supporting requirements;
obtaining deformation of the rock stratum in the underground space by software analysis, and comparing the deformation with a multiple of a size of a tunnel, a value of the multiple of the size of the tunnel being obtained according to use of the underground space and analysis of rock lithology; determining the existing supporting to be effective if the deformation is less than the multiple of the size of the tunnel; and determining the existing supporting to be ineffective and requiring changing of a supporting manner if the deformation is equal to or greater than the multiple of the size of the tunnel; and
determining a supporting length according to the stress environment and the underground surrounding rock structure after the supporting effectiveness is determined;
in step d, a length of an anchor rod for supporting is selected at an interval [0, ½] for the normalized damage degree; and
in step d, when the supporting is ineffective and the supporting manner needs to be changed, supporting density is determined according to a principle that energy E required for prestressing is equal to energy required for multiples of deformation of the underground space, and a value of the multiples of deformation of the underground space is obtained according to use of the underground space and the analysis of rock lithology.

2. The method for determining the supporting structure by combining the stress environment and the underground surrounding rock structure according to claim 1, wherein the laser instrument comprises a shell, and a laser spectroscopy device, a rotating device and a laser scanning device integrally arranged in the shell, wherein the rotating device is configured for rotating the laser scanning device, the laser scanning device is located above the rotating device, and the laser spectroscopy device is located below the rotating device; in actual use, the laser instrument is equipped with a telescopic rod and a placing frame, a front end of the telescopic rod being connected to the laser instrument, and a rear end of the telescopic rod being connected to the placing frame, so that the laser instrument enters an inner wall of a bore hole for detection by adjusting the telescopic rod to obtain the lithology and the thickness of the rock stratum, a rotating speed of the rotating device being matched with a displacement speed of the telescopic rod.

3. The method for determining the supporting structure by combining the stress environment and the underground surrounding rock structure according to claim 2, wherein the laser spectroscopy device comprises a laser source, a focusing lens, a reflecting mirror and a grating, the laser source being configured for reflecting high-energy pulsed laser, the focusing lens being configured for improving capability for detection of the laser at an edge and increasing energy of the laser, the reflecting mirror being configured to refract the high-energy pulsed laser so as to irradiate on a rock surface.

4. The method for determining the supporting structure by combining the stress environment and the underground surrounding rock structure according to claim 1, wherein in step c, comparing the damage degree of the rock stratum at the roof and at the floor is made by selecting rock stratum at different positions at the roof and the floor for comparison according to different lithology.

5. The method for determining the supporting structure by combining the stress environment and the underground surrounding rock structure according to claim 2, wherein the laser scanning device is located above the laser spectroscopy device, the laser scanning device comprises a laser transmitter, a rotatable cylindrical filter, a receiver, a time counter and a CCD camera, wherein the rotating device being located at an end of the laser scanning device close to the laser spectroscopy device, the laser transmitter is configured for emitting infrared laser, and the rotatable cylindrical filter is configured for increasing a laser scanning area, and the laser is reflected back after scanning to the rock, the receiver is configured for receiving a scanning rate, the CCD camera is configured for shooting the rock stratum, and the time counter is configured for controlling the scanning rate, and the laser scanning device rotates 360 degrees under action of the rotating device.

6. A system for identifying an underground surrounding rock structure by using the method for determining the supporting structure by combining the stress environment and the underground surrounding rock structure according to claim 1, comprising a computer, a data transmission line and a laser instrument, wherein the laser instrument comprises a shell, and a laser spectroscopy device, a rotating device and a laser scanning device integrally arranged in the shell, wherein the rotating device is configured for rotating the laser scanning device, the laser scanning device is located above the rotating device, and the laser spectroscopy device is located below the rotating device, and the laser instrument extends into an inner wall of a bore hole for detection to obtain the lithology and the thickness of the rock layer;
the laser scanning device is located above the laser spectroscopy device; the laser scanning device comprises a laser transmitter, a rotatable cylindrical filter, a receiver, a time counter and a CCD camera, the rotating device is located at an end of the laser scanning device close to the laser spectroscopy device, the laser transmitter is configured for emitting infrared laser, and the rotatable cylindrical filter is configured for increasing a laser scanning area, and the laser is reflected back after scanning to the rock, the receiver is configured for receiving a scanning rate, the CCD camera is configured for shooting the rock stratum, the time counter is configured for controlling the scanning rate, and the laser scanning device rotates 360 degrees under action of the rotating device; and
the laser spectroscopy device comprises a laser source, a focusing lens, a reflecting mirror and a grating, wherein the laser source is configured for reflecting high-energy pulsed laser, the focusing lens is configured for improving capability for detection of the laser at an edge and increasing energy of the laser; the reflecting mirror is configured to refract the high-energy pulsed laser so as to irradiate on a rock surface, and excited rock photons are collected by the laser instrument and are transmitted to the computer through the data transmission line, and a spectral image of the rock is formed by software processing in the computer, which is analyzed and compared with data in the database, and a lithology name and spectral characteristics are displayed on a computer screen.
